(19) United States
(12) Patent Application Publication
(10) Pub. No.: US 2002/0128726 A1
(43) Pub. Date: Sep. 12, 2002

(54) Tube encapsulating press on collar for prosthetic leg

(76) Inventor: Kerry E. Wilkinson, Chandler, AZ (US)

Correspondence Address: H. GORDON SHIELDS, 7830 NORTH 23RD AVENUE, PHOENIX, AZ, 85021, US

(21) Appl. No.: 10/142,193

(22) Filed: May 8, 2002

Related U.S. Application Data
(63) Continuation of application No. 09/570,007, filed on May 12, 2000.

(51) Int. Cl. A61F 2/62

(52) U.S. Cl. 623/38

(57) ABSTRACT

A collar includes a counterbore for receiving a rim of a prosthetic leg tube. The collar is press fitted onto the tube, and a pyramid connector element is secured to the collar and the tube to connect the collar and tube to a user. Two different counterbore embodiments in the collar are disclosed, a tapered counterbore and a square counterbore. Set screws extend through aligned tapped apertures in the collar and tube.

### CROSS REFERENCE TO RELATED APPLICATION

This application is a Continuation application of Ser. No. 09/570,007, filed May 12, 2000.

### BACKGROUND OF THE INVENTION

1. Field of the Invention

This invention relates to a prosthetic apparatus and, more particularly, to a collar for a prosthetic leg tube.

2. Description of the Prior Art

Prior art prosthetic pylon units or assemblies include tubes which have a collar at the top for connecting to a prosthetic appliance, a foot or socket, through a pyramid connector element. The pyramid connector includes a curved element and an inverted truncated pyramid lock element. The curved element fits onto the collar, and screws extending through the collar extend to the lock element to secure the tube by its collar to the prosthetic pyramid connector element.

The stress on the collar and tube is, of course, very great, and accordingly the collars are typically of substantial length and are epoxied to the tube. This makes the tube and collar quite long and expensive.

The collar and tube combination is referred to as a pylon. The opposite end of the pylon is typically secured to a socket or to a foot by a clamp assembly.

An alternative to the epoxied collar and tube assembly is a tube and collar assembly machined from a relatively thick walled tubular element. This, obviously, is considerably more expensive than the epoxied tube assembly due to the required machining.

The apparatus of the present invention overcomes the deficiencies of the prior art by providing a relatively inexpensive but strong collar assembly closely integrated with the tube for efficient joining with a prosthetic leg appliance. The attachment screws of the present invention are integrated with the tube and accordingly the long overlap of the prior art is obviated. The prior art requires the long overlap to provide the necessary strength for the unit. Essentially, a major problem of the prior art is simply that the attachment screws are not integrated with the tube, thus requiring the long overlap.

In certain circumstances, a coupling assembly shorter than what is possible in the prior art is required, and the present invention provides such short, and at the same time, strong and relatively inexpensive, coupling assembly.

### SUMMARY OF THE INVENTION

The invention described and claimed herein comprises a collar secured to a tubular element for connecting to a prosthetic appliance through a pyramid connector. The tubular element is encapsulated by the collar. A press fit is used. The collar includes a groove into which an end portion of the tubular element extends. Screws which secure the pyramid connector to the collar and tubular element extend through both the collar and the tubular element.

Among the objects of the present invention are the following:

To provide new and useful prosthetic apparatus;

To provide new and useful collar apparatus for a prosthetic leg;

To provide a new and useful collar, tube, and pyramid assembly for a prosthetic leg in which a set screw extends through both the collar and the tube for securing the pyramid to the collar and the tube;

To provide new and useful prosthetic apparatus having an integrated coupling between a collar and a tube;

To provide new and useful collar and tubular assembly for a prosthetic leg; and

To provide a tube encapsulating press on collar for a prosthetic appliance.

### BRIEF DESCRIPTION OF THE DRAWING

FIG. 1 is a view in partial section of an example of prior art apparatus.

FIG. 2 is a view in partial section of another prior art apparatus.

FIG. 3 is a view in partial section of the apparatus of the present invention in its use environment.

FIG. 4 is a view in partial section of an alternate embodiment of the apparatus of FIG. 3.

### DESCRIPTION OF THE PREFERRED EMBODIMENT

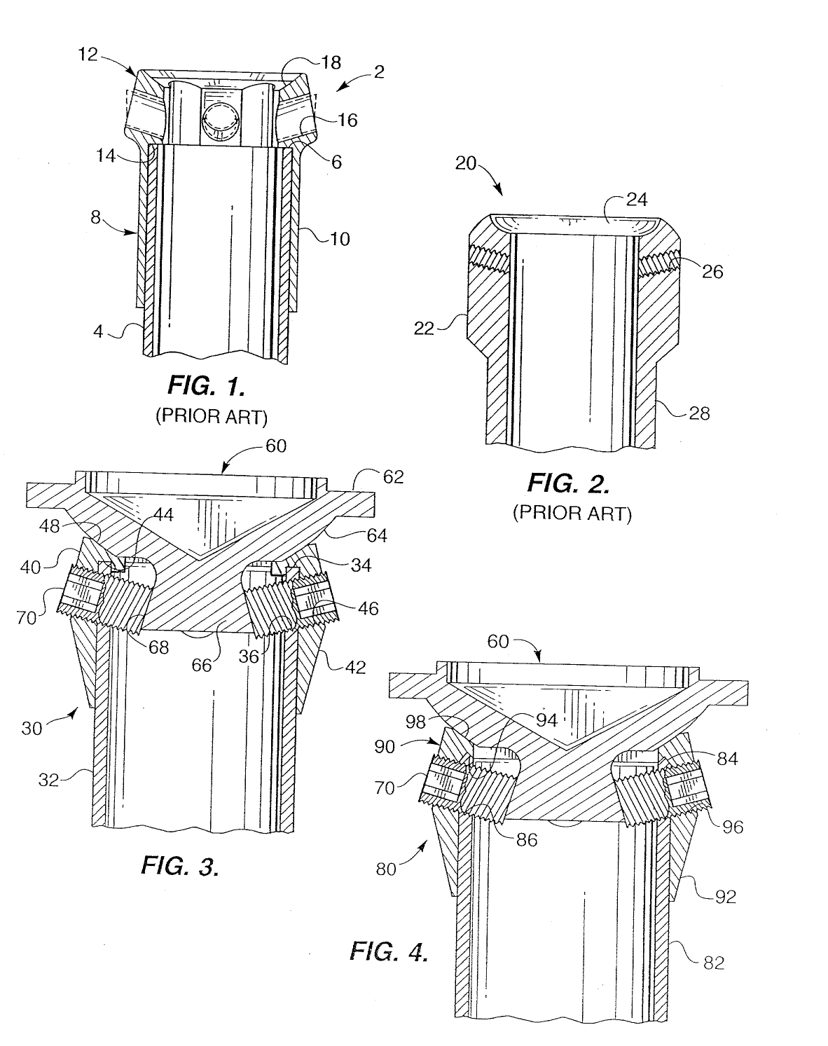

FIGS. 1 and 2 are views in partial section illustrating the prior art. FIG. 1 is a view in partial section through the upper portion of a prosthetic leg pylon 2 which includes a tube 4 with a fitting 8 secured thereto. The tube 4 includes a top 6, and the fitting 8 is disposed about the tube 4 with a top connector portion 12 of the fitting 8 disposed adjacent to the top 6 of the tube 4.

The fitting 8 includes a lower tubular portion 10 which extends for a substantial distance along the tube 4. The connector portion 12 includes a bottom flange 14 which is disposed on the top rim 6 of the tube 4.

Extending through the connector portion 12 are tapped apertures 16. Note that the apertures 16 extend only through the connector portion 12, and not also through the tube 4. A cup portion 18 receives the mating ball portion of a pyramid connector element (see FIGS. 3 and 4) which is used to secure the apparatus 2, as appropriate, to a user's leg, prosthetic foot, etc. Set screws extend through the tapped apertures 16 to secure the pyramid to the apparatus 2.

For securing the tube 4 and the tubular portion 10 together, epoxy is typically used. The epoxy makes a relatively strong bond between the tube 4 and the tubular portion 10. The relatively long overlap of the elements 4 and 10 is needed because of the high stresses when the fitting and pyramid are secured together above the tubular portion 10.

FIG. 2 comprises a view in partial section of another prior art structure 20 in which a tubular element is machined to define an upper fitting portion 22 and a lower tube portion 28. The fitting portion 22 includes a seat on cup portion 24 for receiving the mating portion of a pyramid (see FIGS. 3 and 4). Tapped apertures 26 extend through the upper portion 22 to receive set screws for securing the pyramid to the apparatus 20.

The outer diameter of the fitting portion 22 is the outer diameter of the tube out of which the apparatus 20 is made. The portion of the tube below the fitting 22 is machined to define the tubular section 28. Obviously, the machining process renders the apparatus 20 relatively expensive.

FIG. 3 is a view in partial section of the apparatus of the present invention, comprising a collar and tube apparatus 30. The collar and tube apparatus 30 includes a prosthetic leg tube 32, the upper portion of which is illustrated with a collar 40 secured thereto. A pyramid connector element 60 is shown disposed on the collar 40.

The prosthetic leg tubular element 32 includes an end rim 34 and tapped apertures 36. The collar 40 includes a cylindrical portion 42 through which extends tapped apertures 46. The tapped apertures 46 are appropriately aligned with the tapped apertures 36 which extend through the tube 32.

The collar 40 includes a relatively short tubular portion 42 which fits over the tubular element 32. The collar 40 also includes a counterbore 44. The counterbore 44 receives the rim 34 of the tubular element 32. The counterbore 44 may also be a generally square groove, if desired, instead of a plain square counterbore.

The collar 40 also includes a seat or cup 48 which receives a ball portion 64 of the pyramid 60.

There is a press fit between the tube 32 and the collar 40 to secure the two elements together, with the outer diameter of the tube 32 being substantially the same as the inner diameter of the tubular portion 42 of the collar 40. The width of the counterbore or groove 44 is substantially the same as the wall thickness of the tube 32.

The pyramid connector element 60 includes a flange 62. Disposed beneath the flange 62 is a portion of the ball 64, the upper portion of which is disposed on the seat 48 of the collar 40. Beneath the ball 64 is an inverted pyramid lock element 66, which includes outwardly tapered portions 68. The tapered portions 68 extend slightly outwardly from the juncture of the bottom of the ball 64, and the inverted pyramid lock element 66. Set screws 70 extend through the pairs of aligned tapped apertures 36, and 46 and bear against the tapered portions 68 to secure the pyramid connector element 60 to both the collar 40 and the tube 32 of the apparatus 30.

A pyramid connector 50 is connected to a socket or a prosthetic foot, as is well known and understood in the art.

FIG. 4 comprises a view in partial section of an alternate embodiment of the apparatus of FIG. 3. FIG. 4 illustrates an alternate embodiment 80 which includes a prosthetic leg tube 82, which is substantially the same as the tube 32 of FIG. 3. The prosthetic leg tube 82 includes an end rim 84, and an aperture pair 86 is shown extending through the leg tube 82 below the end rim 84. The rim 84 differs from the rim 36 in that the rim 84 is shown with a tapered or inverted vee configuration.

A collar 90 is shown secured to the upper portion of the tube 82. The collar 90 includes a cylindrical portion 92 through which extends an aperture pair 96. The aperture pair 96 of the cylindrical portion 92 is aligned with the aperture pair 86 of the tube 82. The aligned apertures 86, 96 are tapped, just as the aligned aperture pairs 36 and 44 of FIG. 3 are also tapped, to receive set screws for securing a pyramid connector 60 to the tube and collar apparatus 80. Thus, the pyramid 60 is directly connected to both the tube 82 and the collar 90.

The collar 90 also includes a tapered or vee shaped groove 94 which receives the tapered rim 84 of the tube 82.

Again, the collar 90 and the tube 82 include a press fit to secure the collar 90 to the tube 82. The press fit securely holds the tube 82 and the collar 90 together, along with the set screws 70 in the aligned and tapped apertures 86 and 96, as with the apparatus 30.

It will be noted that the inner diameter of the cylindrical portions 42 and 92 of the collars 40 and 90, respectively, are substantially the same as the outer diameter of the tubes 32 and 82, as referred to. This, of course, provides the press fit required to help secure the elements together. Moreover, the maximum width of the tapered or vee shaped groove 94 is substantially the same as the wall thickness of the tube 82, just as the width of the counterbore or groove 44 of the collar 40 is substantially the same as the thickness of the tube 32. Accordingly, the counterbores or grooves also help in the press fit between the respective collars and their respective tubes.

The press fit in the embodiments of FIGS. 3 and 4 helps secure the collars to the tubes, and the fact that the set screws extend through both the collars and the tubes assures a secure connection between the tubes, the collars, and the pyramids such that relatively strong yet relatively inexpensive pylons result therefrom.

The collar 90 includes a seat 98 which receives the mating ball portion of the pyramid 60, as with the apparatus 30 of FIG. 3.

While the principles of the invention have been made clear in illustrative embodiments, there will be immediately obvious to those skilled in the art many modifications of structure, arrangement, proportions, the elements, materials, and components used in the practice of the invention, and otherwise, which are particularly adapted to specific environments and operative requirements without departing from those principles. The appended claims are intended to cover and embrace any and all such modifications, within the limits only of the true spirit and scope of the invention.

What I claim is:

1. Prosthetic leg apparatus comprising in combination:
a prosthetic leg tube;
a collar including a cylindrical portion having an inner diameter substantially the same as the outer diameter of the prosthetic leg tube;
counterbore means in the cylindrical portion for receiving the prosthetic leg tube; and
aperture means extending through the cylindrical portion of the collar and through the prosthetic leg tube for receiving a set screw for securing the collar and the prosthetic leg tube to a pyramid connector.

2. The apparatus of claim 1 in which the counterbore means has a generally square configuration.

3. The apparatus of claim 1 in which the counter bore means includes a tapered groove for receiving the prosthetic leg tube.

4. The apparatus of claim 1 in which the counterbore means has a width that is substantially the same as the wall thickness of the prosthetic leg tube.

5. The apparatus of claim 1 which includes a seat for receiving a ball of a pyramid connector element for connecting the tube, the collar, and the pyramid connector together.

6. The apparatus of claim 1 in which the aperture means includes aligned and tapped apertures in the collar tube.

7. Prosthetic leg apparatus comprising in combination:
a tubular element having a wall thickness, and outer diameter, and a rim;
collar means secured to the tubular element for connecting the tubular element to a pyramid connector element, including
a cylindrical portion having an inner diameter which is substantially the same as the outer diameter of the tubular element,
a counterbore for receiving the rim of the tubular element; and
aperture means extending through the collar means and the tubular element for receiving a set screw to secure a pyramid connector element to the collar means and the tubular element.

8. The apparatus of claim 7 in which the collar means further includes a seat for receiving a portion of the pyramid connector element.

9. The apparatus of claim 7 in which the aperture means includes aligned apertures in the collar means and the tubular element.

10. The apparatus of claim 9 in which the aligned apertures are tapped to receive the set screw.

11. The apparatus of claim 7 in which the counterbore includes a tapered groove.

12. The apparatus of claim 7 in which the counterbore means comprises a square shaped counterbore.

13. Prosthetic leg apparatus for connecting to a pyramid connector comprising in combination:
a prosthetic leg tube;
a collar having a cylindrical portion;
a counter bore in the cylindrical portion; and
a groove in the counter bore for receiving a portion of the prosthetic leg tube.

14. The apparatus of claim 13 in which the groove is tapered.

15. The apparatus of claim 13 which further includes a seat in the collar for receiving a portion of a pyramid connector.

16. The apparatus of claim 15 which further includes aligned apertures in the collar and the prosthetic leg tube for receiving a set screw for securing together the collar, the prosthetic leg tube, and the pyramid connector.
